(19) United States
(12) Patent Application Publication
(10) Pub. No.: US 2011/0124025 A1
(43) Pub. Date: May 26, 2011

(54) Cell Collecting Devices and Methods for Collecting Cells

(75) Inventors: James CASTRACANE, Albany, NY (US); Waseem Khan RAJA, Latham, NY (US)

(73) Assignee: COLLEGE OF NANOSCALE SCIENCE AND ENGINEERING, Albany, NY (US)

(21) Appl. No.: 12/623,294

(22) Filed: Nov. 20, 2009

(51) Int. Cl. C12Q 1/02; C12M 1/00; C12N 5/00; A61B 10/02

(52) U.S. Cl. 435/29; 435/287.1; 435/404; 600/562

(57) ABSTRACT

A cell collecting device having a housing with an inlet for receiving cells, a cell attractant cavity have a cell attractant, a cell collection channel running from the inlet to the cell attractant cavity, and a plurality of electrodes positioned to detect the presence of cells is disclosed. The cell attractant cavity may include a porous medium, such as, a hydrogel, containing the cell attractant, such as, epidermal growth factor. The channel may include a plurality of restrictions and expansions to assist in maintaining the porous medium while permitting the passage of attractant and cells. The device may be implanted into a patient for an extended period of time, and then removed and examined. A method for collecting cells, an implantable attractant dispersing device, and a porous medium for controlled releasing of a compound are also disclosed.

### FEDERAL FUNDED RESEARCH

The invention described herein was made with U.S. Government support under Grant Number U54-CA126511 awarded by the National Institutes of Health (NIH). The U.S. Government has certain rights to this invention.

### BACKGROUND

1. Field of the Invention

The present invention relates to microfluidic devices having microsensors, and more particularly, to implantable microfluidic devices adapted to collect biological substances, for example, cancer cells, for extraction and examination.

2. Description of Related Art

Controlled cell growth and division is an indication of normal, healthy cells. Cells in human and animal organs constantly interact with their environment, that is, their “microenvironment,” and this microenvironment includes cell behavior and cell gene expression. The microenvironment of a tumor is complex and can play a critical roll in the invasion or metastasis of tumor cells to adjacent vessels and tissue. Accordingly, there is a need in the art to examine the microenvironment of tumors and their cells in order to better understand cell behavior and movement, that is, chemotaxis, and, it is hoped, fashion effective remedies.

According to the conventional art, cancer cells may typically be extracted from the tumor or its vicinity for external, that is, ex vivo, examination and testing. The investigations performed by Condeelis, et al. (2000) showed that motile cancer cells can be attracted and captured by inserting a needle bearing growth factor adjacent to the cells. The cells are attracted to the growth factor, for example, epidermal growth factor (EGF), and captured by the needle. The growth factor, or chemoattractant, may be embedded in a substrate that retains the chemoattractant, for example, a protein matrix, such as, Matrigel protent matrix provided by BD Biosciences, or its equivalent. According to the prior art, the chemoattractant diffuses into the surrounding tissue forming a chemoattractant gradient that attracts motile cells into the syringe or catheter whereby the cells can be collected in the syringe or catheter and extracted with the syringe or catheter.

However, this prior art method of extracting cancer cells from a patient has inherent disadvantages. For example, the relatively short intervals of cell collection from the tumor provide little information about the longer-term dynamics of the microenvironment of the tumor. In particular, for a better understanding of the behavior of tumor cells and their motility, the information provided by such short-term cell extraction is limited, and may be ineffective in providing worthwhile information concerning, for example, cancer cell metastasis. A method and device for collecting cancer cells, in vivo, for extended periods of time could be critical to understanding cancer cell motility and thus lead to minimization or prevention of cancer cell metastasis. Aspects of the present invention provide methods and devices that address this need.

### BRIEF SUMMARY OF ASPECTS OF THE INVENTION

A first aspect of the invention is a cell collecting device comprising or including a housing having an inlet for receiving cells; a cell attractant cavity positioned in the housing distal the inlet, the attractant cavity have a cell attractant, for example, EGF or CSF; a cell collection channel having a proximal end in fluid communication with the inlet and a distal end in fluid communication with the attractant cavity, the collection channel comprising a plurality of collection chambers positioned to receive cells from the inlet; and a plurality of electrodes positioned in the collection channel and adapted to contact the cells collected therein, wherein contact with the cells provides a detectable variation in an electrical property, for example, impedance, across at least two of the plurality of electrodes. In one aspect, the plurality of collection chambers comprise varying widths, for example, varying in width in a direction from the cell attractant cavity to the inlet.

In another aspect, the device further comprises a porous medium positioned in at least the attractant cavity, the porous medium containing the cell attractant. The porous medium may be a porous silicon, a porous hydrogel, or a porous protein. The porous medium may comprise a PEGDA hydrogel or a blend of PEGDA and PEGMA hydrogel, for example, a blend containing about 10% to about 30% PEGDA and about 0.5% to about 15% PEGMA, for instance, typically, a hydrogel containing about 18% to about 22% PEGDA and about 8% to about 12% PEGMA.

In another aspect, the housing of the collecting device may include a cover having an aperture and a rupturable membrane positioned in the aperture. The rupturable membrane may be used to discharge cells from the collection device after extraction from a patient, for example, by applying an overpressure to the inlet, which ruptures the membrane and discharges at least some of the cells.

Another aspect of the invention is a method of collecting cells comprising or including positioning a device having a housing with an inlet for receiving cells, a cell attractant cavity positioned in the housing distal the inlet, the attractant cavity having a cell attractant, a cell collection channel having a proximal end in fluid communication with the inlet and a distal end in fluid communication with the attractant cavity; allowing the cell attractant to flow from the attractant cavity, through the cell collection channel, and out of the inlet; attracting at least some cells with the cell attractant into the inlet and at least partially into the channel; detecting the presence of the cells in the channel; wherein allowing the cell attractant to flow through the channel comprises restricting a flow of attractant through the channel by providing a plurality of restrictions in the channel. In one aspect, allowing the cell attractant to flow through the channel further comprises, after restricting the flow of attractant through at least one of the plurality of restrictions, allowing the flow to expand into one of a plurality of expansion cavities in the channel.

In another aspect of the invention, the method includes regulating the flow of attractant from the attractant cavity, for example, by providing a porous medium containing the attractant into the attractant cavity. The porous medium containing the cell attractant may be a porous silicon or a porous hydrogel. The porous medium may be a biodegradable medium, a bio-erodable medium, or a non-biodegradable medium; for example, the porous medium may be a biodegradable polymer, a bio-erodable polymer, or a non-biodegradable polymer, or a combination thereof. Also, regulating the flow of attractant from the attractant cavity may be practiced by varying the concentration of the one or more polymers of the hydrogel, for example, varying the concentration of the one or more cross-linking polymers of the hydrogen, for instance, the PEGDA and/or PEGMA in the hydrogel of the porous medium.

A further aspect of the invention is an implantable attractant dispersing device comprising or including a housing having an outlet for attractant and an attractant cavity; and a porous medium containing attractant positioned in the attractant cavity and adapted to release attractant out of the outlet. The porous medium may be made from a porous silicon and/or a porous hydrogel. For example, the porous medium may be a hydrogel having PEGDA and/or PEGMA.

A still further aspect of the invention is a porous medium for releasing a compound at a desired release rate comprising a hydrogel comprising at least one of PEGDA and a blend of PEGDA and PEGMA. The porous medium may comprise a hydrogel comprising a blend of about 10% to about 30% PEGDA and about 0.5% to about 15% PEGMA. In one aspect, a hydrogel containing about 18% to about 22% PEGDA and about 8% to about 12% PEGMA may be provided. The compound released may be a chemoattractant, for example, EGF, CSF, or a combination thereof.

Aspects of the invention are marketed under the name NANIVID (that is, NANo-Intra-VIital-Device). Aspects of the invention may also be gleaned from prior publications, for example, Raja, et al. “The NANIVID: A New Device for Cancer Cell Migration Studies,” Proceedings of SPIE Volume: 6859 pp. 68591M-68591M-8, 3008 [herein “Raja (2008)”]; Raja, et al. “A new diagnostic for cancer dynamics: Status and initial test of the NANIVID,” Proceedings of SPIE Volume: 7207, pp. 72070E-1 to 72070E-8, 2009 [herein Raja (2009)]; Borocan, A. J., a master's thesis entitled, “NANIVID: a New Technology for Cancer Studies,” College of Nanoscale Science and Engineering, 2009 [herein “Borocan (2009)]; and pending U.S. application Ser. No. 10/945,563 filed on Sep. 20, 2004 [attorney ref 0794.050A]. The disclosure of these references are incorporated by reference herein in their entirety.

These and other aspects, features, and advantages of this invention will become apparent from the following detailed description of the various aspects of the invention taken in conjunction with the accompanying drawings.

### BRIEF DESCRIPTION OF THE DRAWINGS

The subject matter, which is regarded as the invention, is particularly pointed out and distinctly claimed in the claims at the conclusion of this specification. The foregoing and other objects, features, and advantages of the invention will be readily understood from the following detailed description of aspects of the invention taken in conjunction with the accompanying drawings in which:

FIG. 1 is perspective view of a collecting device according to one aspect of the invention.

FIG. 2A is a perspective view of the base of the device shown in FIG. 1 with the cover removed.

FIG. 2B is a perspective view of the inverted cover of the device shown in FIG. 1 with the base removed.

FIG. 3 is a top plan view of another aspect of the invention similar to the device shown in FIG. 1.

FIG. 4 is a front elevation view of the device shown in FIG. 3

FIG. 5 is a side elevation view of the device shown in FIG. 3.

FIGS. 6 through 15 are top plan views of collecting devices according to further aspects of the invention.

FIG. 16 is a top plan view of a photomicrograph of a collecting device according to one aspect of the invention.

FIGS. 17 and 18 present chemical formulas of two polymers that may be used in the production of a porous medium according to one aspect of the invention.

FIG. 19 represents the polymerization reaction of the polymer shown in FIG. 17 to produce a porous medium according to one aspect of the invention.

FIGS. 20, 21, and 22 are scanning electron microscope images (SEM) of porous media that may be used in aspects of the invention.

FIGS. 23 and 24 are a bar chart and a set of curves, respectively, of the results of testing of cell attractant release according to aspects of the invention.

### DETAILED DESCRIPTION OF ASPECTS OF THE INVENTION

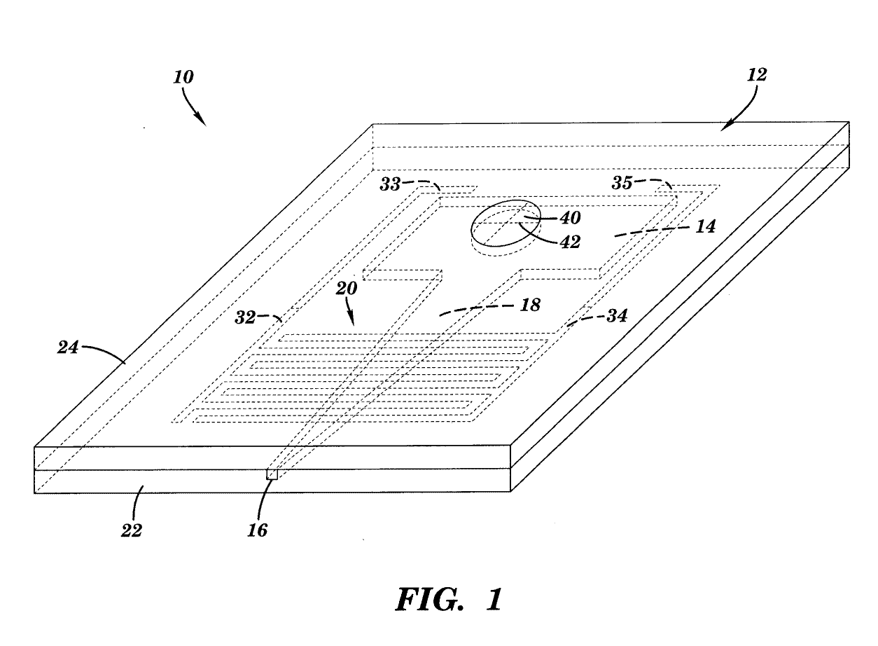

FIG. 1 is perspective view of collection device 10 according to one aspect of the invention. Collection device 10 may be a medical device that may be used to collect any compounds or structures, for example, cells, that, for example, respond to an attractant, for example, a chemoattractant. Though aspects of the invention are specifically adapted to be used in the medical field, more specifically, to oncological testing and analysis, aspects of the invention are not limited to use in the medical field, but may be used in any field where the collection of minute particles or bodies is worthwhile.

In the following discussion of the invention, cells, for example, cancer cells, will be used almost exclusively for the type of structures collected by device 10, however, it will be understood that the use of the term “cell” or “cells” is only meant to facilitate description of aspects of the invention, which are not limited to the collection of cells. In some aspects of the invention, compounds, structures, and/or fluids may be collected, for example, biological an/or non-biological compounds, structures, and/or fluids, for example, cells, biological and non-biological chemicals and chemical compounds, macrophages, fibroblasts, bacteria, or any other compounds or structures that may be encountered in mammalian or non-mammalian bodily tissue and/or fluids. For example, in one aspect, aspartic acid may be used to attract bacteria into device 10.

In another aspect of the invention, device 10 may also be adapted to release, expel, dose, or distribute compounds, structures, and/or fluids to a surrounding environment, for example, for treatment of tissue or dosing of medication. According to aspects of the invention, the compounds, structures, and or fluids released may include biological an/or non-biological compounds, structures, and/or fluids, for example, cells, biological and non-biological chemicals and chemical compounds, macrophages, fibroblasts, bacteria, particles, microparticles, nanoparticles, medication, treatments, or any other compounds, structures, and/or fluids that may be encountered in mammalian or non-mammalian bodily fluids or desirably released to mammalian or non-mammalian bodily tissue and/or fluids.

As shown in FIG. 1, device 10 comprises a housing 12 containing a cavity 14, an inlet 16, a channel or passage 18 between cavity 14 and inlet 16, and a plurality of electrodes 20 exposed to channel 18. Cavity 14, channel 18, electrodes 20 are typically completely enclosed in housing 12 and are therefore shown in phantom in FIG. 1. Though inlet 16 may provide for ingress of cells, inlet 16 may also function as and be referred to as an “outlet” in some aspects of the invention. Though housing 12 may comprise a single integral structure, as shown in FIG. 1, housing 12 may typically include a base 22 and a cover 24 mounted to base 22.

According to aspects of the invention, a compound that attracts cells, that is, a cell attractant (such as, epidermal growth factor (EGF)) is preloaded into cavity 14, that is, the attractant cavity, and allowed to flow, for example, by diffusion and/or gravity and/or capillary action, into and through channel 18. According to the invention, cells exposed to the attractant adjacent to inlet 16 are attracted to and migrate (for example, via chemotaxis) though inlet 16 and into channel 18. In one aspect, the cell attractant is allowed to escape from cavity 14 and flow through channel 18 and the cell attractant may be discharged from inlet 16. According to one aspect, a gradient of the concentration of cell attractant is provided between cavity 14 and inlet 16 that promotes migration or chemotaxis of cells into inlet 16 and toward cavity 14. According to another aspect of the invention, at least some form of sensing means is provided to detect the presence of cells in channel 18, for example, two or more electrodes 20 positioned to detect the presence of cells in channel 18. The number of cells that may be collected may range from less than 10 to 10s of thousands; the number of cells collected may generally be dependent upon the length of time device 10 is implanted in a patient. The length of time for which embodiments of the invention are implanted and collecting cells may vary from minutes, to hours, to days, to weeks, to months, and, in some aspects, to years.

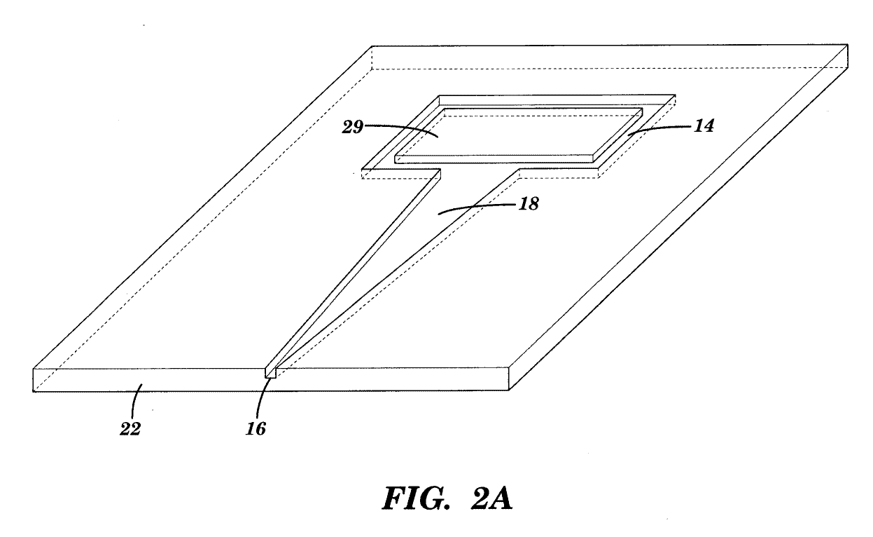

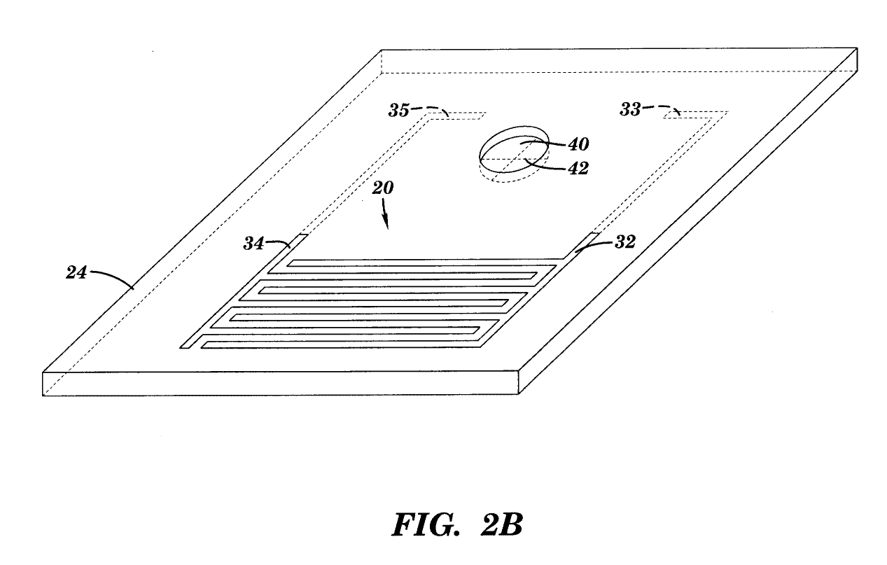

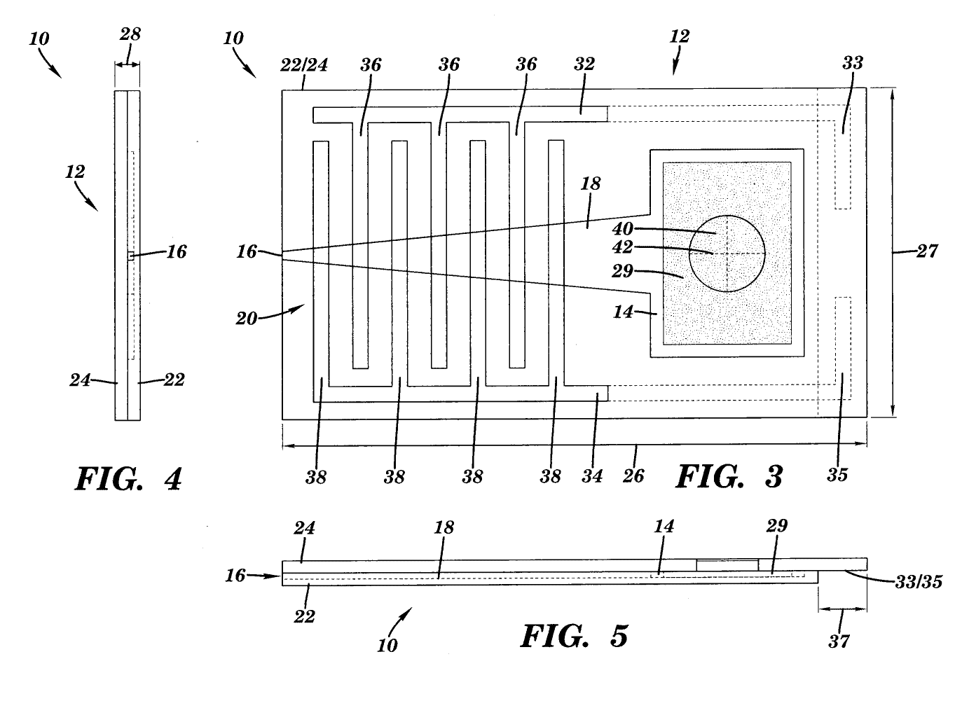

FIG. 2A is a perspective view of base 22 of device 10 shown in FIG. 1 with cover 24 removed and FIG. 2B is a perspective view of inverted cover 24 of device 10 shown in FIG. 1 with base 22 removed to more clearly illustrate features of the invention. FIG. 3 is a top plan view of device 10 shown in FIG. 1, but, as discussed below, slightly modified to have exposed electrodes. FIG. 4 is a front elevation view of device 10 shown in FIG. 3 and FIG. 5 is a side elevation view of device 10 shown in FIG. 3.

In one aspect of the invention, the cell attractant in attractant cavity 14 show in FIG. 2A may be infused into a porous medium or “sponge” adapted to retain and subsequently release the cell attractant. As shown in FIGS. 2A through 5, cavity 14 may include a porous medium 29 containing cell attractant. As will be discussed below, the porous medium 29 may comprise, a structure, a body, or a “sponge” 29 uniquely fabricated to provide a desired release of cell attractant to channel 18 to enhance operation of device 10. In one aspect, at least some attractant may also be placed in channel 18, for example, the porous medium 29 containing attractant may be positioned in cavity 14 and/or at least some porous medium 29 containing attractant may be positioned in channel 18.

As discussed above, embodiments of the invention may comprise minute devices that, for example, are intended to be embedded into a subject, for example, a human or an animal, to collect cells or release a substance. Accordingly, embodiments of the invention may vary broadly in size. For example, according to aspects of the invention, housing 12 shown in FIGS. 1 through 5 may have a length 26 ranging from about 100 micrometers (μm) to about 50 millimeters (mm) and typically will have a length 26 between about 1 mm about 3 mm. Housing 12 may have a width 27 ranging from about 1000 μm to about 5 mm, and typically will have a width 27 between about 1 mm and about 3 mm, and housing 12 may have a thickness 28 ranging from about 100 μm to about 2 mm and typically will have a thickness 28 of between about 500 μm and about 1000 μm.

Accordingly, embodiments of the invention may be manufactured by using conventional semiconductor fabrication methods, for example, photolithographic methods, including deposition, masking, and selective etching, among others. Device 10 may be fabricated from conventional plastics, metals, or conventional semiconductor materials. However, in one aspect device 10 is transparent to the electromagnetic radiation used to examine device 10 while implanted in a patient, that is, in vivo, where cells can be viewed without being obscured by device 10. For example, when device 10 is viewed in vivo using visible light, for example, viewed by multi-photon microscopy, housing 12 may preferably be transparent to visible light. Similarly, when device 10 is being examined in vivo with x-rays, device 10 may preferably be fabricated from materials transparent to x-rays. In one aspect, the base 22 of housing 12 may be fabricated from silicon, but may typically be fabricated from glass, for example, from tempered soda-lime glass, such as, Pyrex® glass, marketed by Corning, or its equivalent. Base 22 may have a thickness of 1000 μm or less, for example, 100 μm or less. Cover 24 may also be made from any conventional material, for example, silicon or glass. In one aspect, cover 24 may be made from glass cover slips provided by Thermo-Fisher Scientific. Cover 24 may have a thickness of 1000 μm or less, for example, 100 μm or less.

Cavity 14 and channel 18 may be fabricated in base 22 and/or cover 24 by conventional photolithographic methods. In one aspect of the invention, at least a portion of cavity 14 and/or channel 18 may be provided in cover 24 with a complementary portion of cavity 14 and channel 18 provided in base 22. However, in one preferred aspect, the bottom surface and side walls of cavity 14 and channel 18 are formed in base 22 and the top surface of cavity 14 and channel 18 are provided by cover 24 when assembled.

In the aspect of the invention shown in FIGS. 1 through 5, cavity 14 is shown as a generally rectangular cavity, and channel 18 is shown as a generally convergent channel with generally planar convergent sidewalls. However, in other embodiments of the invention, cavity 14 and channel 18 may assume a broad range of shapes and geometries without departing from the operation and function of the invention. For example, cavity 14 may be circular in shape, or elliptical or triangular in shape, and have an overall dimensions consistent with the general dimensions of device 10 disclosed above. For instance, FIGS. 7-16 illustrate a generally circular cavity 14. Similarly, some examples of the broad range of shapes of channel 18 according to aspects of the invention are shown in FIGS. 7-16.

Regardless of their shape, cavity 14 and channel 18 may be fabricated by conventional forming processes, for example, molding, casting, or machining; however, due to their size, cavity 14 and channel 18 may typically be fabricated by conventional photolithographic methods, for example, cleaning, masking, etching, and stripping. Cavity 14 and channel 18 may have flat or rounded bases or floors. Accordingly, the shape of inlet 16, which may comprise an extension of channel 18, may be defined by the shape of channel 18 and may be circular or non-circular in cross section. For example, inlet 16 may be square or rectangular in cross section as shown in FIGS. 1 and 2. The depth of cavity 14 and channel 18 may be the same or vary. For example, the floor or base of cavity 14 may be above or below the floor or base of channel 18. The depth of cavity 14 and the depth of channel 18 from the top surface of base 20 may vary from about 20 μm to about 500 μm, and may typically be from about 50 μm to about 70 μm. In one aspect, two or more cavities 14 and channels 18 may be formed in a single base or substrate 22, for example, 10 or more or 100 or more may be formed in a single substrate by conventional photolithographic processes.

As mentioned above, according to another aspect of the invention, at least some form of sensing means is provided to detect the presence of cells in channel 18. For example, a sensing means may be provided to indicate to the investigator that cells have entered channel 18 as desired, or that a certain number or a volume of cells have entered channel 18, whereby device 10 may be removed from the patient for analysis and evaluation of the cells collected. According to one aspect of the invention, any mechanism may be used to detect the presence of cells in channel 18. These mechanisms may be mechanical means, for example, weight, mass, or deflection detection; chemical means, for example, consumption of reactant or detection of the heat of reaction; or electrical means, for example, a detection of some electrical property or characteristic of device 10 that is indicative of the presence or absence of cells in channel 18.

In one aspect of the invention, electrical means is provided in device 10 to detect the presence of cells in channel 18. Again, a variety of electrical components can be used to detect the presence of cells in channel 18. (For example, see Borocan, A. J., a master's thesis entitled, “NANIVID: a New Technology for Cancer Studies,” College of Nanoscale Science and Engineering, 2009, which is incorporated by reference herein in its entirety, for a discussion of electrical detection methods that may be used in aspects of the invention.) As shown in FIGS. 1 through 5, according to one aspect, a plurality of electrodes 20, or an electrode array, may be positioned in device 10 where at least a portion of the electrodes 20 are positioned to contact cells entering channel 18. In one aspect, at least a portion of two electrodes 20 may be positioned adjacent to or within channel 18 whereby the presence of cells in channel 18, for example, in contact with electrodes 20, produces a variation in an electrical property that can be detected and/or sensed via contact electrodes 32 and 34. As shown in FIG. 2B, in one aspect, electrodes 20 may be positioned on the surface of cover 24 whereby, when cover 24 shown in FIG. 2B is inverted and positioned on base 22, for example, shown in FIG. 2A, at least some of the electrodes 32 and 34 span channel 18 in base 22 whereby cells collected in channel 18 may contact electrodes 32 and 34 and provide a detectable variation in an electrical property.

As shown in FIG. 3, contact electrodes 32 and 34 may be positioned on device 10 wherever convenient, for example, as indicated in phantom by alternate contact electrodes 33 and 35. As shown in FIGS. 3 and 5, in one aspect, electrodes 33 and 35 on cover 24 may be at least partially exposed whereby electrical contact with external contacts may be made with electrodes 33 and 35. For example, at least some of the base 22 may be removed, for example, etched away, to expose electrodes 33 and 35. As shown in FIGS. 3 and 5, in one aspect, cover 24 may be longer in length (or width) than base 22 by a dimension 37 (see FIG. 5) whereby electrodes 33 and 35 are exposed for external contact.

According to aspects of the invention, any variation in electrical characteristic that may be sensed at electrodes 32 and 34 may be used to detect the presence of cells. The variation of one or more of the following electrical parameters may be used to provide an indication of the presence of cells in channel 18: voltage, current, resistance, capacitance, inductance, impedance, and/or power. The electrical characteristic detected may be transmitted to an external and/or remote receiver, for example, by means of an external contact or wires hardwired to contacts 32 or wirelessly transmitted from contacts 32. In one aspect of the invention, a transmitter may be provided on device 10, for example, a transmitter electrically coupled to contacts 32, 34. The transmitter may be a radio frequency (RF) transmitter among other transmitting devices, for example, among other electromagnetic energy transmitting devices, may be used. The external receiver may perform data manipulation to provide meaningful data, and may be, for example, a data acquisition system or computer.

In one aspect, the inventors have found that a variation in impedance across contact electrodes 32, 34 may be used to detect the presence of or increased or decreased presence of cells in channel 18. For example, the inventors have found that the accumulation of cells in channel 18 varies the impedance across electrodes 32, 34. Specifically, the accumulation of cells on or adjacent to electrodes 20 varies the impedance (typically, increases the impedance) across contact electrodes 32, 34 and this impedance and its variation can be detected, for example, by means of an impedance or voltage meter. (See Borocan (2009) for a discussion of the variation of impedance with cell accumulation.) Other means of electrically detecting the presence of cells in channel 18 will be apparent to those of skill in the art while residing within the scope of the present invention.

In one aspect, a plurality of interdigitally positioned electrodes 20 may be used to detect a variation in electrical property across contact electrodes 32, 34. As shown in FIGS. 1, 2A, 2B, and 3, electrodes 20 may comprise a series of interdigitally positioned electrodes 36 and 38, where electrodes 36 are in electrical communication with electrical contact 32 and electrodes 38 are in electrical communication with electrical contact 34. In the aspect of the invention shown in FIG. 3 there are three electrodes 36 and four electrodes 38; however, the number of electrodes 36 and 38 may vary from one to 10, to 20 or more per contact electrode 32, 34. In addition, the spacing, length, and shape of electrodes 36 and 38 may vary without departing from the scope of the invention. (See Borocan (2009) for a discussion of the impact of electrode spacing.)

Electrodes 20, 32, 33, 34, 35, 36, and 38 may also be fabricated by conventional or photolithographic means. The electrodes may be fabricated from any conductive material for example, copper (Cu), silver (Ag), gold (Au), chromium (Cr), or titanium (Ti), among other electrical conductors. However, in one aspect, where it is preferred that device 10 and its components be transparent to the radiation being used to monitor or examine device 10, the electrodes may be made from a material transparent to the radiation used. For example, when visible light is used, electrodes 20, 32, 33, 34, 35, 36, and 38 may be fabricated from indium-doped tin oxide (ITO), or its equivalent, which is transparent to visible light. Again, electrodes 20, 32, 33, 34, 35, 36, and 38 may be provided on base 22 by conventional methods (for example, physical vapor deposition (PVD) and like methods) or by conventional photolithographic methods, for example, cleaning, spin-on resist, developing, deposition, and resist lift off, among other processes. Electrodes 20, 32, 33, 34, 35, 36, and 38 may typically have a thickness from about 50 nm to about 200 nm and a width from about 500 nm to about 100 μm.

As shown in FIGS. 1, 2B, 3, and 5 cover 22 may be provided with a ruptural membrane 40 in a vicinity of cavity 14. Membrane 40 may be positioned adjacent one of the surfaces of cover 24, for example, whereby membrane 40 is flush with either surface of cover 24, though membrane 40 may be positioned below either surface of cover 40. According to aspects of the invention, membrane 40 may be provided to facilitate removal of the collected cells after completion of the collection. For example, upon detection from electrodes 20 that an appropriate number or volume of cells were collected in channel 18, device 10 can be removed from the patient, for example, surgically or via catheter, and the collected cells examined. In one aspect, the contents of device 10 may be dislodged from device 10 by the application of an over-pressure to inlet 16 sufficient to rupture membrane 40 and discharges at least some, preferably all, of the cells collected by device 10. Membrane 40 may be fashioned to assist its rupture by an applied pressure, for example, membrane 40 may be fashioned sufficiently thin to rupture under a predetermined pressure, or membrane 40 may be weakened by the addition of stress concentrators, for example, one or more score lines 42, to enhance the likelihood of rupture. Membrane 40 may also be provided by conventional fabrication methods or by conventional photolithographic methods, such as, selective etching. Membrane 40 may be circular, as shown, or non-circular, for example, square, rectangular, or elliptical, and may typically have a thickness from about 500 nm to about 1000 nm and a width or diameter from about 100 μm to about 1000 μm.

After formation of cavity 14, channel 18, and electrodes 20, and the insertion of porous medium 29 into cavity 14, cover 22 is applied to base 20 to complete the formation of cavity 14 and chancel 18 and provide a sealed enclosure for device 10. Cover 22 may be applied to base 20 with any conventional adhesive. However, as noted above, to ensure transparency, cover 22 may be adhered or bonded to base 20 using a transparent adhesive, such as, a hydrogel, for example, the hydrogel used to provide porous medium 29, or a polydimethylsiloxane PDMS adhesive. With the addition of cover 22 on base 20, the fabrication of device 10 may be substantially complete. As is typical in the art, two or more devices 10 may be fabricated on a single substrate or base 20 and then separated into separate devices 10 by conventional separation means, for example, conventional cutting or dicing. Each device 10 may typically be inspected for structural accuracy and the electrodes calibrated for subsequent use.

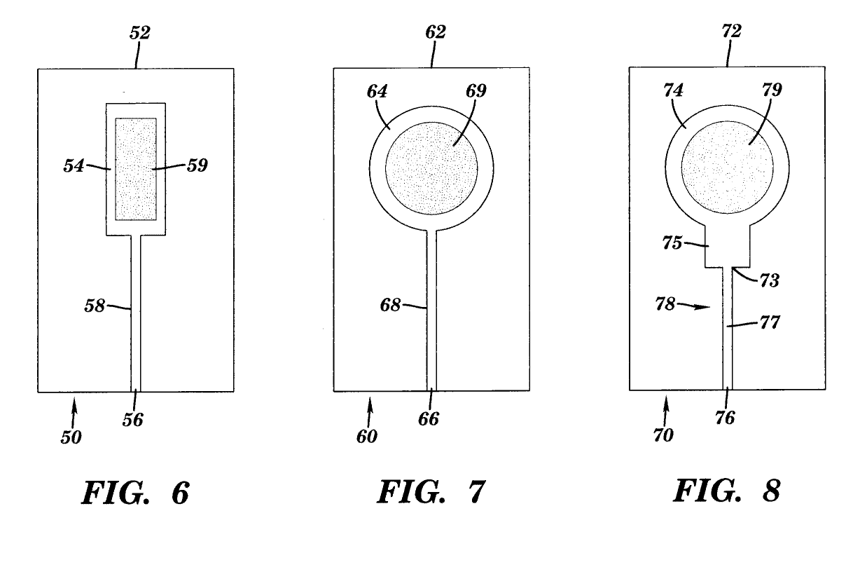

FIGS. 6 through 15 are top plan views of collection devices according to further aspects of the invention with their covers 24 removed. The devices shown in FIGS. 6 through 15 may have all the features and dimensions of device 10 shown in FIGS. 1 through 5. The principle differences between device 10 shown in FIGS. 1-5 and the devices shown in FIGS. 6 through 15 are the shapes of the attractant cavities and collection channels shown in FIGS. 6 through 15. The cell sensing device, for example, electrodes 20, are omitted from FIGS. 6-15 to clarify the aspect shown, though some form of cell sensing device and associated electrodes may typically be present in the devices shown in FIGS. 6-15.

FIG. 6 is a top plan view of a collection device 50 having a base 52, an attractant cavity 54, a collection channel 58, and an inlet 56. As shown, cavity 54 is generally rectangular in shape and collection channel 58 comprises a substantially uniform, elongated channel from cavity 54 to inlet 56. A cell attractant may typically be provided in a porous medium 59 and allowed to flow from cavity 54 through channel 58.

FIG. 7 is a top plan view of a collection device 60 having a base 62, an attractant cavity 64, a collection channel 68, and an inlet 66. As shown, cavity 64 is generally circular in shape and collection channel 68 comprises a substantially uniform, elongated channel from cavity 64 to inlet 66. A cell attractant may typically be provided in a porous medium 69 and allowed to flow from cavity 64 through channel 68.

FIG. 8 is a top plan view of a collection device 70 having a base 72, an attractant cavity 74, a collection channel 78, and an inlet 76. As shown, cavity 74 is generally circular in shape and collection channel 78 comprises a first wider passage 75 adjacent to cavity 74 and abrupt transition 73 to a second narrow passage 77. Passage 77 is substantially a uniform, elongated channel leading to inlet 76. A cell attractant may typically be provided in a porous medium 79 and allowed to flow from cavity 74 through channel 78 to inlet 76.

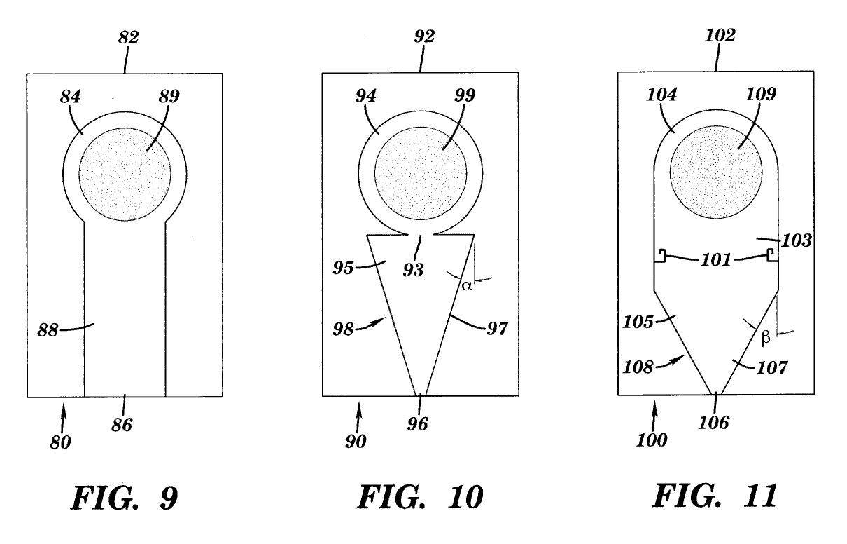

FIG. 9 is a top plan view of a collection device 80 having a base 82, an attractant cavity 84, a collection channel 88, and an inlet 86. As shown, cavity 84 is generally circular in shape and collection channel 88 comprises a substantially uniform, elongated channel, wider than channel 68 in FIG. 7, from cavity 84 to inlet 86. A cell attractant may typically be provided in a porous medium 89 and allowed to flow from cavity 84 through channel 88.

FIG. 10 is a top plan view of a collection device 90 having a base 92, an attractant cavity 94, a collection channel 98, and an inlet 96. As shown, cavity 94 is generally circular in shape. Collection channel 98 comprises a restriction 93 and then expands in a converging channel 95 having converging sidewalls 97 that lead to inlet 96. Sidewalls 97 may converge at an angle α (alpha) that may range from 30 degrees to 60 degrees. A cell attractant may typically be provided in a porous medium 99 and allowed to flow from cavity 94 through channel 98 to outlet 96.

FIG. 11 is a top plan view of a collection device 100 having a base 102, an attractant cavity 104, a collection channel 108, and an inlet 106. As shown, cavity 104 is generally circular in shape. Collection channel 108 comprises a first elongated section 103 substantially as wide as the diameter of cavity 104 and then and a second converging section 105 having converging side walls 107 that lead to inlet 106. Sidewalls 107 may converge at an angle β (beta) that may range from 30 degrees to 60 degrees. A cell attractant may typically be provided in a porous medium 99 and allowed to flow from cavity 94 through channel 98. Device 100 may include one or more retaining structures, obstructions, or “hooks” 101 that assist in retaining the porous medium or “sponge” 99 in attractant cavity 104.

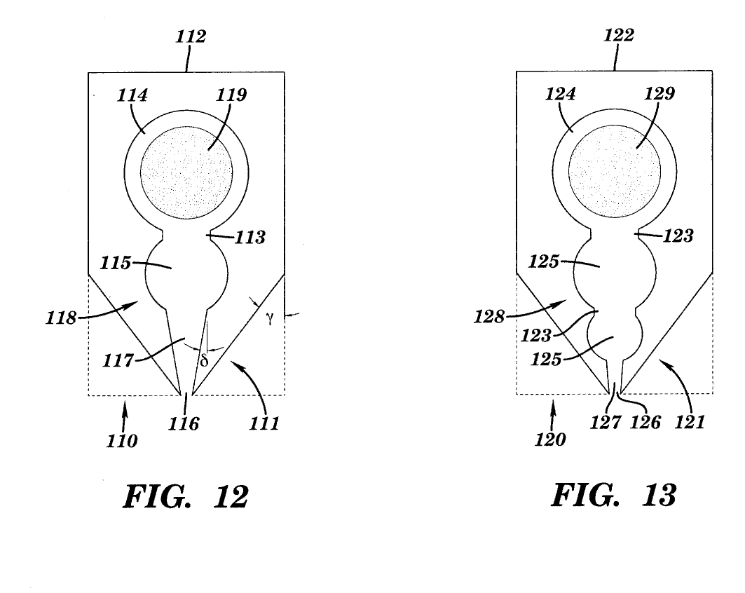

FIG. 12 is a top plan view of a collection device 110 having a base 112, an attractant cavity 114, a collection channel 118, and an inlet 116. In contrast to earlier aspects of the invention, base 112 includes a converging portion 111, though as shown in phantom no convergence may be provided, at the end of base 112 adjacent to inlet 116. According to aspects of the invention, this convergent portion 111 or “point” may assist the investigator in inserting device 110 into the patient. It is to be understood that any of the devices disclosed herein may have a convergent section similar to portion 111. Convergent portion 111 of base 112 may converge at an angle γ (gamma) that may range from 30 degrees to 60 degrees.

Cavity 114 of device 110 in FIG. 12 is generally circular in shape. Collection channel 118 comprises a restriction 113 between cavity 114 and at least one first collection cavity or expansion 115. Collection cavity 115 may typically be narrower in expanse, for example, smaller in width or diameter, than attractant cavity 114. Channel 118 also includes an outlet from cavity 115 to a converging channel 117 that leads to inlet 116. Sidewalls of channel 117 may converge at an angle δ (delta) that may range from 5 degrees to 30 degrees; the sidewalls of channel 117 may also be substantially parallel or divergent. A cell attractant may typically be provided in a porous medium 119 and allowed to flow from cavity 114 through channel 118 to inlet 116. The inventors have found that providing one or more collection cavities 115 enhances the performance of device 110, for example, by limiting the ingress of undesirable bodily fluids in to channel 118 and cavity 114 while permitting the flow of cell attractant from cavity 114. Though collection cavity 115 is shown as generally circular in FIG. 12, the one or more collection cavities 115 that may comprise collection channel 118 may be circular or non-circular, for example, square, rectangular or elliptical.

FIG. 13 is a top plan view of a collection device 120 having a base 122, an attractant cavity 124, a collection channel 128, and an inlet 126. Base 122 includes a converging portion 121, though as shown in phantom no convergence may be provided, at the end of base 122 adjacent to inlet 126, similar to converging portion 111 shown in FIG. 12, and have all the attributes of convergent portion 111.

Cavity 124 of device 120 in FIG. 13 is generally circular in shape. Collection channel 128 comprises two or more restrictions 123, similar to restriction 113 in FIG. 12, and two or more collection cavities 125, similar to cavities 115 shown in FIG. 12, between cavity 124 and inlet 126. Again, collection cavities 125 may typically be narrower in expanse, for example, smaller in width or diameter, than attractant cavity 124, and may progressively become narrower (though in one aspect, they may become wider) as channel 128 approaches inlet 126. Channel 128 also include an outlet from cavity 125 to a converging channel 127 that leads to inlet 126. The sidewalls of channel 127 may converge at an angle that may range from 1 degree to 20 degrees; the sidewalls of channel 127 may also be substantially parallel or divergent. A cell attractant may typically be provided in a porous medium 129 and be allowed to flow from cavity 124 through channel 128 to outlet 126. Again, the inventors have found that providing one or more collection cavities 125 enhances the performance of device 110. Though collection cavity 125 is shown as generally circular in FIG. 13, the two or more collection cavities 125 that may comprise collection channel 128 may be circular or non-circular, for example, square, rectangular or elliptical. The shapes of collection cavities 125 may also vary in shape, for example, one or more circular, one or more square or rectangular.

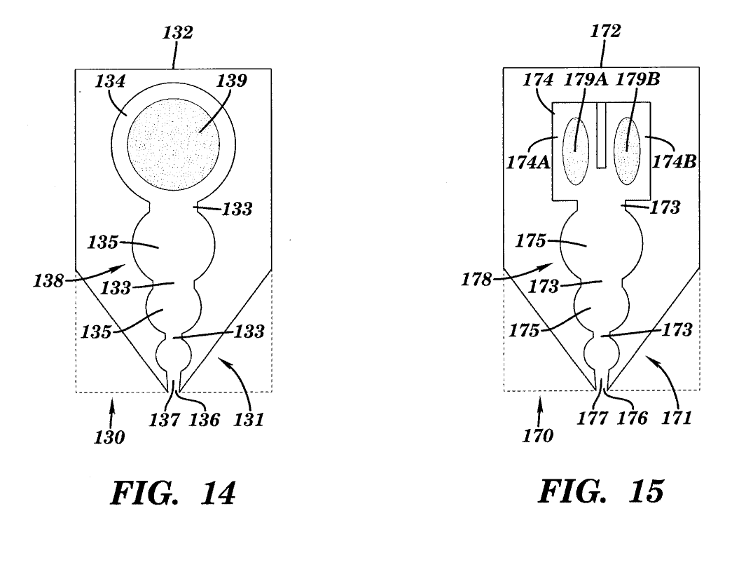

FIG. 14 is a top plan view of a collection device 130 having a base 132, an attractant cavity 134, a collection channel 138, and an inlet 136. Base 132 includes a converging portion 131, though as shown in phantom no convergence may be provided, at the end of base 132 adjacent to inlet 136, similar to converging portion 111 shown in FIG. 12, and having all the attributes of convergent portion 111.

Cavity 134 of device 130 in FIG. 14 is generally circular in shape. Collection channel 138 comprises three or more restrictions 133, similar to restriction 113 in FIG. 12, between cavity 134, and three or more collection cavities or expansion cavities 135, similar to cavities 115 shown in FIG. 12. Again, collection cavities 135 may typically be narrower in expanse, for example, smaller in width or diameter, than attractant cavity 134, and may progressively become narrower (thought in one aspect, they may become wider) as channel 138 approaches inlet 136. Channel 138 also includes an outlet from a cavity 135 to a channel 137 that leads to inlet 136. The sidewalls of channel 137 may converge at an angle that may range from 1 degrees to 15 degrees; the sidewalls of channel 137 may also be substantially parallel or divergent.

A cell attractant may typically be provided in a porous medium 139 and be allowed to flow from cavity 134 through channel 138 to inlet 136. Again, the inventors have found that providing one or more collection cavities 135 enhances the performance of device 130. Though collection cavities 135 are shown as generally circular in FIG. 14, the three or more collection cavities 135 that may comprise collection channel 138 may be circular or non-circular, for example, square, rectangular or elliptical. The shapes of collection cavities 135 may also vary in shape, for example, one or more circular, one or more square or rectangular.

FIG. 15 is a top plan view of a collection device 170 having a base 172, an attractant cavity 174, a collection channel 178, for example, similar to collection channel 138 shown in FIG. 14, and an inlet 176. Base 172 includes a converging portion 171, though as shown in phantom no convergence may be provided, at the end of base 172 adjacent to inlet 176, similar to converging portion 111 shown in FIG. 12, and having all the attributes of convergent portion 111.

As shown in FIG. 15 attractant cavity 174 may comprise two or more attractant cavities 174A and 174B holding two or more porous media 179A, 179B. The two or more attractant cavities 174A, 174B may typically be in fluid communication with channel 178, for example, in direct, parallel communication with channel 178. In one aspect, the two or more attractant cavities 174A, 174B may be in indirect fluid communication with channel 178, for example, in series. Though cavities 174A and 174B may be rectangular as shown, cavities 174A and 174B may be circular, square, or oval among other shapes. Similar to channel 138 shown in FIG. 14, collection channel 178 may comprise three or more restrictions 173, similar to restriction 113 in FIG. 12, between cavity 174 and inlet 176, and three or more collection cavities or expansion cavities 175, similar to cavities 115 shown in FIG. 12. Again, collection cavities 175 may typically be narrower in expanse, for example, smaller in width or diameter, than attractant cavity 174, and may progressively become narrower (though in one aspect, they may become wider) as channel 178 approaches inlet 176. Channel 178 also includes an outlet from a cavity 175 to a channel 177 that leads to inlet 176. The sidewalls of channel 177 may converge at an angle that may range from 1 degrees to 15 degrees; the sidewalls of channel 177 may also be substantially parallel or divergent.

A cell attractant or a substance to be released may typically be provided in the porous media 179A, 179B and be allowed to flow from the two or more cavities 174A and 174B, through channel 178 to inlet 176. Though collection cavities 176 are shown as generally circular in FIG. 15, the three or more collection cavities 175 that may comprise collection channel 178 may be circular or non-circular, for example, square, rectangular or elliptical. The shapes of collection cavities 175 may also vary in shape, for example, one or more circular, one or more square or rectangular.

According to the aspect of the invention shown in FIG. 15, the two or more attractant cavities 174A, 174B may contain porous media 179A, 179B, containing one or more attractants or substances to be released. For example, according to aspects of the invention, a plurality of cavities 174A, 174B containing one or a plurality of attractants or other substances for release to channel 178 may be provided, for example, one or more different attractants or substances. In addition, the characteristics of the porous media 179A, 179B may vary, for example, to provide varying release of the attractant or other substance contained in the porous media 179A, 179B. It will be understood that the inventors envision that the multiple cavities 1774A, 174B having porous media 179A, 179B containing one o more attractants or substances shown in FIG. 15 may be used in any of the aspects of the invention disclosed herein, for example, in any aspect illustrated in FIGS. 1-16.

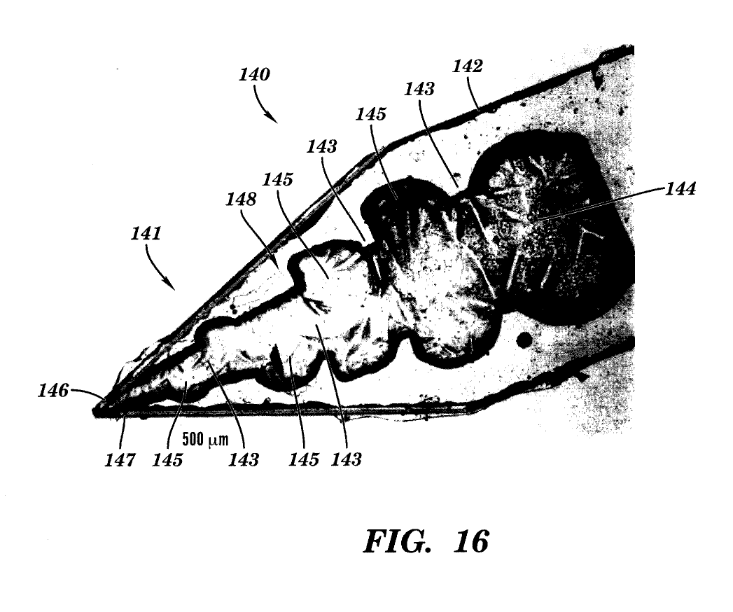

FIG. 16 is a partial top plan view of a photomicrograph of an actual cell collecting device 140 according to one aspect of the invention. Collection device 140 includes a base 142, an attractant cavity 144, a collection channel 148, and an inlet 146. Base 142 includes a converging portion 141 at the end of base 142 adjacent to inlet 146, similar to converging portion 111 shown in FIG. 12, and having all the attributes of convergent portion 111. A plurality of sensing electrodes and transmitting electrodes may typically also be present, for example, as shown by electrodes 20 in FIGS. 1-5, but are not shown in FIG. 16.

As shown in FIG. 15, attractant cavity 144 of device 140 is generally circular in shape, but the shape of cavity 144 may deviate from purely circular, square, or rectangular, due to the inaccuracies or tolerances of the fabrication process, for example, the photolithographic processes. Collection channel 148 comprises approximately four (4) restrictions 143, similar to restriction 113 in FIG. 12, and approximately five (5) collection cavities or expansion cavities 145, similar to cavities 115 shown in FIG. 12, between cavity 144 and inlet 146. Collection cavities 145 may generally be circular in shape, but, again, the shape of cavities 145 may deviate from purely circular, square, or rectangular, due to the inaccuracies or tolerances of the fabrication process, for example, the photolithographic processes. As shown, collection cavities 145 may typically be narrower in expanse, for example, smaller in width or diameter, than attractant cavity 144, and may progressively become narrower as channel 148 approaches inlet 146. Channel 148 also includes an outlet from a cavity 145 to a channel 147 that leads to inlet 146. The sidewalls of channel 147 may converge or diverge at an angle or, as shown, may be substantially parallel.

A porous medium having an attractant may typically be located in attractant cavity 144 and/or in channel 148, but is not shown in FIG. 16. Again, the inventors have found that providing one or more collection cavities 145 enhances the performance of device 140.

According to aspects of the invention, the channels 18, 58, 68, etc., illustrated in FIG. 1-16 provide cavities into which cells may enter and collect for subsequent examination after extraction of the devices disclosed herein. However, in one aspect, the movement or retention of cells in channels 18 etc. may be enhanced by providing a retaining structure in these channels, for example, a 3-dimensional mesh, structure, or medium that may enhance the adherence of cells to channel 18 or enhance the movement of cells along channel 18. In one aspect, this retaining structure may be provided by Matrigel or a hydrogel, for example, one of the hydrogels disclosed herein. Other hydrogels, retaining structures, or movement enhancing structures will be apparent to those of skill in the art.

As discussed previously, according to aspects of the invention, a cell attractant is released from the attractant cavity, for example, cavity 14 in FIGS. 1-5, and allowed to flow through the collection channel 18 toward inlet 16. The attractant, for example, epidermal growth factor (EGF), colony stimulatory factor (CSF), or a combination thereof, among others, is released from cavity 14 to provide the presence of the attractant or a concentration gradient of the attractant to stimulate the attraction of target cells, for example, motile cells, from the vicinity of inlet 16 into channel 18 to collect or capture the cells for subsequent examination. In order to effectively provide a consistent, timely release of attractant, in one aspect, it is preferred to at least partially control or partially regulate the release of attractant to channel 18 and out of inlet 16. For example, it may be undesirable for the attractant to rapidly flow from cavity 14 and out of inlet 16 whereby the attractant is dispersed quickly. In one aspect of the invention, the attractant is dispersed over a specified time period, for example, hours, days, weeks, months, or years to provide a relatively consistent attractant concentration level in channel 18 and/or in the vicinity of inlet 16. The controlled release of attractant can help to ensure that the desired attraction of cells is maintained over the desired time period. According to aspects of the invention, at least some control of the release of attractant from cavity 14 is provided by embedding the attractant into a porous medium or mass 29 (in FIGS. 1-5) that releases the reactant, for example, via diffusion, over a desired period of time. For example, the interaction of the pores in the medium with the viscosity of the attractant may at least partially limit the flow of attractant from the porous medium to channel 18 and inlet 16.

According to one aspect of the invention, any porous material may be used for porous medium 29 which is compatible with the size and dimensions of device 10 and cavity 14. Porous medium 29 may be made from an organic or an inorganic material, a biodegradable medium or a non-biodegradable medium. For example, in one aspect of the invention, a porous silicon (Si) may be used, where the attractant is introduced to the porous silicon either prior to or after inserting the porous silicon into cavity 14. In another embodiment, the porous material may comprise a gelatinous protein mixture embedded with attractant, for example, a protein mixture marketed under the name Matrigel protein by BD Biosciences, or its equivalent. In another embodiment, the porous medium 29 may be a porous polymer, for example, a biodegradable polymer, a bio-erodable polymer, a non-biodegradable polymer, or a combination thereof. The porous medium 29 may be fabricated by combining two or more polymers, for example, a bio-degradable polymer, a bio-erodable polymer, a non-biodegradable polymer, or a combination thereof, and then dissolving one of the polymers with a solvent to leave one of the polymers with a series of pores once filled by the dissolved polymer. For example, SU-8 photoresist may be mixed with poly(methyl methacrylate) (PMMA) and cured. When the PMMA is dissolved with a suitable solvent, the remaining porous SU-8 provides a matrix into which a attractant can be embedded.

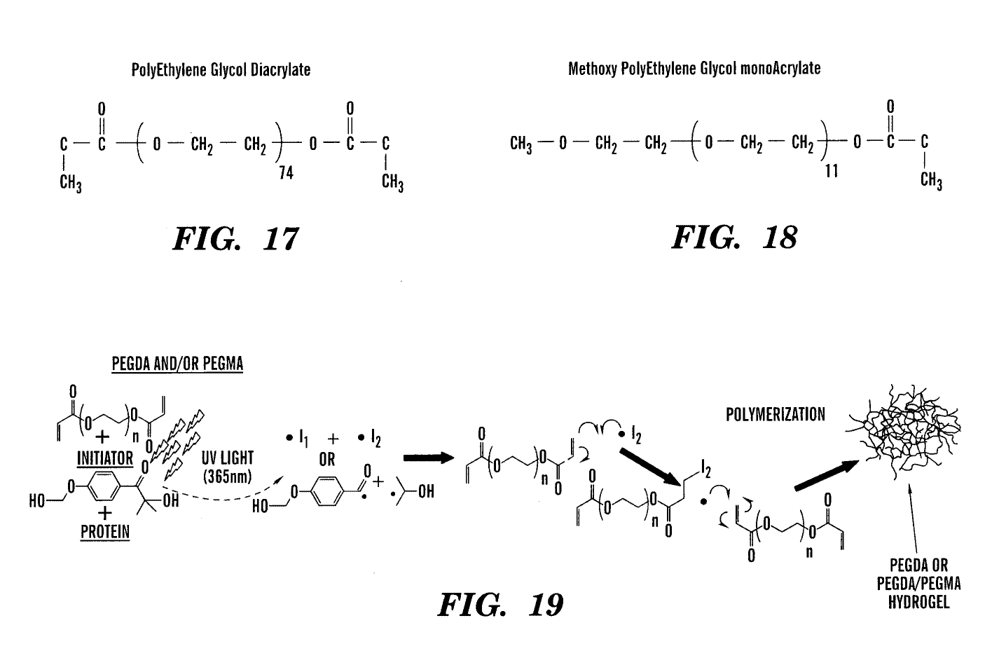

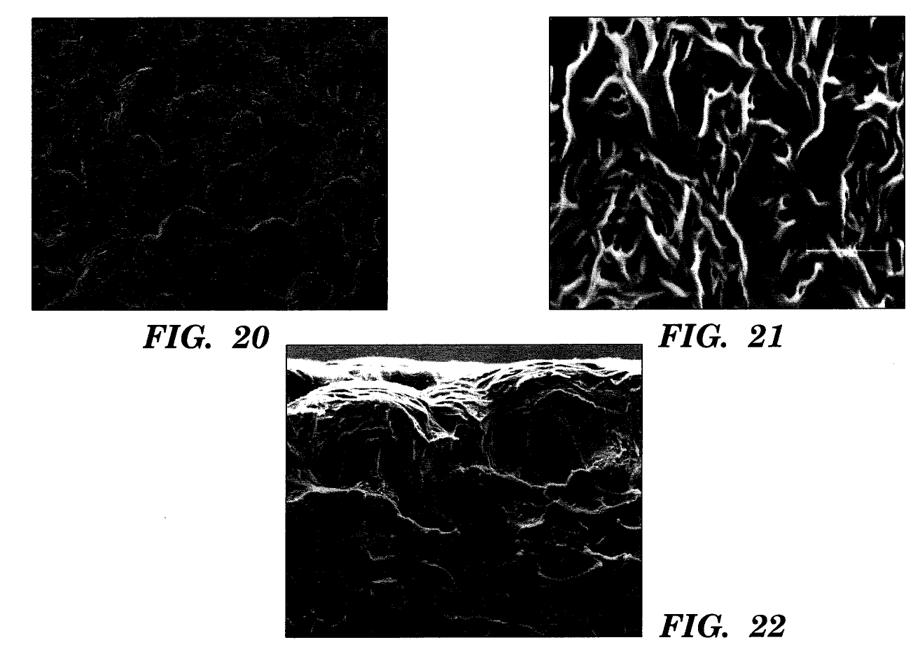

In another aspect of the invention, the porous material may comprise a “hydrogel,” that is, a water-soluble, absorbent network of polymer chains. Though according to aspects of the invention the hydrogel used for the porous medium may be fabricated by any conventional means of making a hydrogel, for example, from a bio-degradable polymer, a bio-erodable polymer, a non-biodegradable polymer, or a combination thereof, in one aspect, the hydrogel may be fabricated from a suitably treated cross-linking agent. For example, in one aspect, the hydrogel for the porous medium may be fabricated from polyethylene glycol diacrylate (PEGDA) or a blend of PEGDA and a methoxy polyethylene glycol monoacrylate (PEGMA), or their equivalents. According to one aspect of the invention, a “blend” of polymers may be provided in which two or more species may be provided in a mixture, though other polymer and/or chemical species may be present. In preliminary testing, these hydrogels were selected due to their biocompatibility. FIGS. 16 and 17 present the chemical formulas of PEGDA and PEGMA, respectively, that may be used in the production of a porous medium according to one aspect of the invention. The cross-linking agent, such as, PEGDA and/or PEGMA, may be provided in powder form and/or a liquid form (for example, PEGDA may typically be provided as a powder and PEGMA may typically be provided as a liquid) and may be dissolved in an appropriate solvent, for example, phosphate buffer saline (PBS), to a desired cross-linking agent concentration. A curing agent, for example, Irgacure-2969 photo-initiator for UV curing provided by Ciba Corporation, and the cell attractant may be introduced to cross-linking agent/solvent solution. The solution of cross-linking agent, solvent, photo-initiator, and attractant may be cured with UV light, for example, at 365 nm. Other curing agents and reaction vehicles may also be used. FIG. 19 illustrates the UV photo-initiation and polymerization reaction of PEGDA cross-linking agent with a attractant (identified as the “protein” in FIG. 18) to produce a PEGDA-hydrogel porous medium having the desired cell attractant according to one aspect of the invention. FIGS. 20, 21, and 22 are scanning electron microscope images (SEM) of actual porous PEGDA hydrogels that may be used for the porous medium 29 in aspects of the invention. The porosity of the polymer matrix is clearly indicated by the voids in the polymer matrices shown.

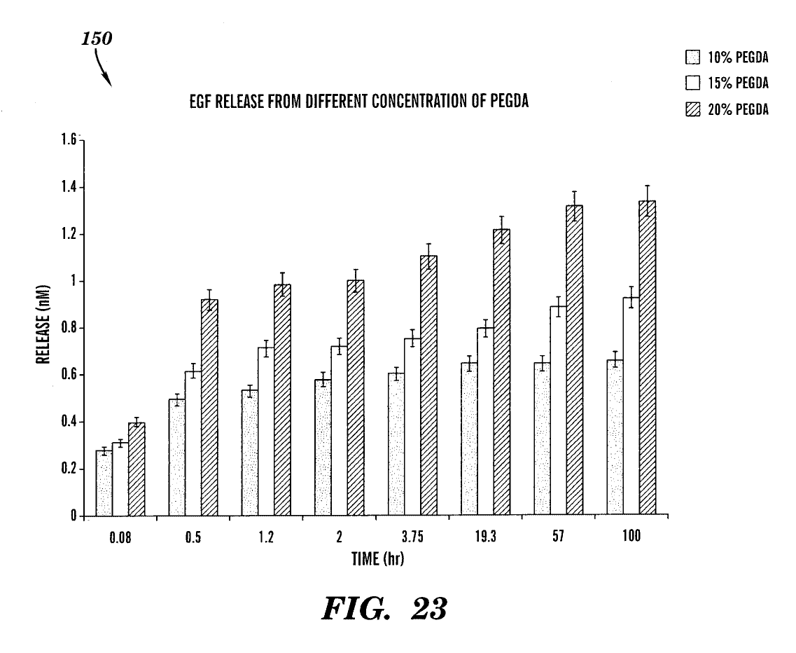

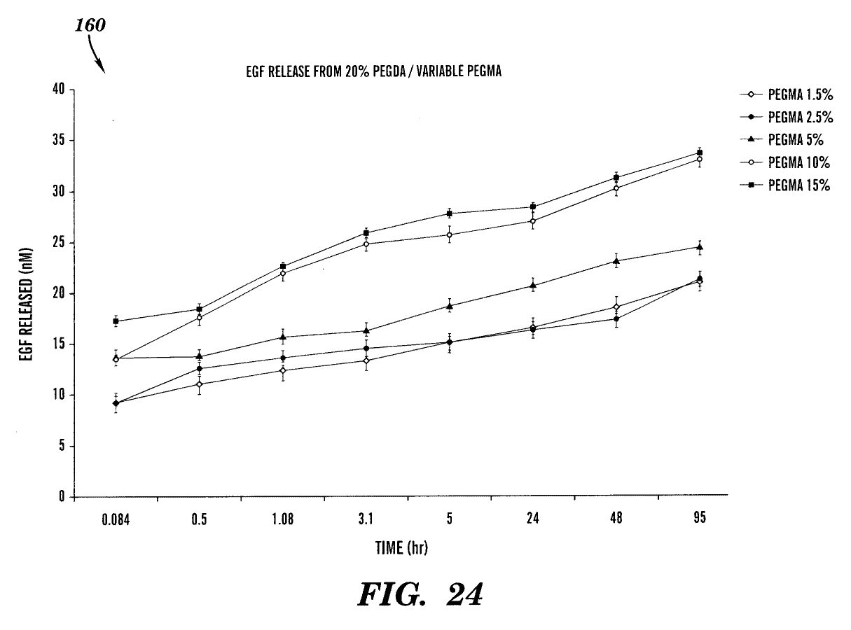

The inventors investigated the attractant release rates of porous hydrogel matrices according to aspects of the invention. In one set of experiments, three concentrations of PEGDA hydrogels (10%, 15%, and 20%) were cured in the presence of EGF. The details of the experimentation are documented in Raja, et al. “A new diagnostic for cancer dynamics: Status and initial test of the NANIVID,” (2009), the disclosure of which is incorporated by reference herein in its entirety. FIGS. 23 and 24 are a bar chart 150 and curves 160, respectively, of the results of release time testing of PEGDA hydrogels and a blend of PEGDA-PEGMA hydrogels, respectively, with EGF as the cell attractant according to aspects of the invention. The abscissa of both FIGS. 23 and 24 is time, in hours; the ordinate in both FIGS. 23 and 24 is amount of EGF released, in nanoMolar (nM).

As shown in FIG. 23, as the concentration of PEGDA increases from 10% to 20% the rate and amount of EGF released from the PEGDA hydrogel increases. For example, the 20% PEGDA hydrogel released more EGF than the 10% PEGDA hydrogel and the 20% PEGDA hydrogel released the EGF more rapidly than the 10% PEGDA hydrogel. Moreover, the amount and rate of release appear to be relatively proportional to the concentration of PEGDA in the hydrogel. Among other things, this indicates that the rate and amount of attractant released may be tailored to a desired release rate. For example, higher concentrations of PEGDA may be used for more rapid release and lower concentrations of PEGDA may be used for slower release of attractant.

As shown in FIG. 24, similar results of the testing of EGF release from hydrogels containing 20% PEGDA and varying percentages of the concentration of PEGMA are summarized. In these tests, the composition of the PEGDA in the hydrogel was held constant and the concentration of the PEGMA was varied from 1.5%, to 2.5%, to 5%, to 10% and to 15%. As shown by curves 160, the amount of EGF released from the blend of PEGDA-PEGMA hydrogels increases with increased PEGMA concentration. For example, the 20% PEGDA-15% PEGMA hydrogel released more EGF at a faster rate than any other 20% PEGDA-PEGMA hydrogel. Moreover, the amount and rate of release appears to be relatively proportional to the concentration of PEGMA in the hydrogel. Among other things, this again indicates that the rate and amount of attractant released may be tailored to a desired release rate. For example, higher concentrations of PEGMA with PEGDA may be used for more rapid release of attractant while lower concentrations of PEGMA with PEGDA may be used for slower release of attractant.

Accordingly, in one aspect of the invention, a PEGDA hydrogel and/or a blend of PEDGA/PEDMA hydrogel are provided with a characteristic time release of attractant, for example, for use in the devices defined herein. In another aspects of the invention, a PEGDA hydrogel and/or a blend of PEDGA/PEDMA hydrogel are provided having a time release of an attractant or a similar compound in any application where time release of an attractant or similar compound is desired.

The porous mass embedded with attractant, for example, a PEGDA hydrogel embedded with EGF or a blend of PEGDA/PEGMA hydrogel as described above, may be introduced or “loaded” to attractant cavity 14 in device 10, or any of the other devices disclosed herein, by syringe to provide aspects of the present invention.

According to aspects of the invention, the devices provided may typically maintain their efficacy for as long as desired from manufacture to point of use. For example, embodiments of the invention may have a sufficient “shelf life” where they can be fabricated, handled, packaged, stored, unstored, and prepared for use without degrading the efficacy of the collection, for example, cell collection, and/or delivery desired, for example, without degrading the efficacy of the attractant. For example, devices according to aspects of the invention may typically have a shelf life of at least hours, but typically, days, weeks, months, or even years. In one aspect, the shelf life of a device according to aspects of the invention can be enhanced through refrigeration or freezing, for example, the efficacy of embodiments of the invention may be preserved by cooling, for example, to a temperature of at least about 0 zero degrees C., but typically to at least about minus 20 degrees C., while retaining their efficacy when prepared for use.

It will be apparent to those of skill in the art that aspects of the invention provide devices and methods for collecting substances, such as cells and related matter, from patients from localized areas, for example, near or in tumors, organs, blood vessels, etc, or anomalous structures. Aspects of the invention may be used for collection of cells over extended periods to study the time dependent behavior of, for example, cancer cells, that is simply unavailable in the prior art. Though some aspects of the invention are uniquely provided for use in medical investigations, other aspects of the invention are not limited to medicine, but may be used wherever the collection of minute particles or bodies is worthwhile.

While several aspects and embodiments of the present invention have been described and depicted herein, alternative aspects and embodiments may be provided by those skilled in the art to accomplish the same objectives. Accordingly, it is intended by the appended claims to cover all such alternative aspects and embodiments as fall within the true spirit and scope of the invention.

1. A cell collecting device comprising;
a housing having an inlet for receiving cells;
a cell attractant cavity positioned in the housing distal the inlet, the attractant cavity have a cell attractant;
a cell collection channel having a proximal end in fluid communication with the inlet and a distal end in fluid communication with the attractant cavity, the collection channel comprising a plurality of collection chambers positioned to receive cells from the inlet; and
a plurality of electrodes positioned in the collection channel and adapted to contact the cells collected therein, wherein contact with the cells provides a detectable variation in an electrical property across at least two of the plurality of electrodes.

2. The device as recited in claim 1, wherein the plurality of collection chambers have varying widths.

3. The device as recited in claim 2, wherein the varying widths of the plurality of collection chambers vary in a direction from the cell attractant cavity to the inlet.

4. The device as recited in claim 3, wherein the varying widths of the plurality of collection chambers decrease in the direction from the cell attractant cavity to the inlet.

5. The device as recited in claim 1, wherein the device further comprises a porous medium positioned in at least the attractant cavity, the porous medium containing the cell attractant.

6. The device as recited in claim 5, wherein the porous medium comprises at least one of a porous silicon and a porous hydrogel.

7. The device as recited in claim 5, wherein the porous medium comprises a PEGDA hydrogel.

8. The device as recited in claim 5, wherein the porous medium comprises a PEGDA/PEGMA hydrogel.

9. The device as recited in claim 8, wherein the PEGDA/PEGMA hydrogel comprises about 10% to about 30% PEGDA and about 0.5% to about 15% PEGMA.

10. The device as recited in claim 1, wherein the housing comprises a cover, and wherein the cover includes an aperture and a rupturable membrane positioned in the aperture.

11. The device as recited in claim 10, wherein the rupturable membrane is adapted to rupture upon application of an over pressure to the inlet, and wherein at least some of the cells received by the collection chambers are discharged through the aperture.

12. The device as recited in claim 1, wherein the device further comprises means for transmitting the detectable variation in the electrical property to a remote receiver.

13. The device as recited in claim 1, wherein the electrical property comprises impedance.

14. The device as recited in claim 1, wherein the cell attractant comprises at least one of EGF and CSF.

15. A method of collecting cells comprising:
positioning a device having a housing with an inlet for receiving cells, a cell attractant cavity positioned in the housing distal the inlet, the attractant cavity have a cell attractant, a cell collection channel having a proximal end in fluid communication with the inlet and a distal end in fluid communication with the attractant cavity;
allowing the cell attractant to flow from the attractant cavity, through the cell collection channel, and out of the inlet;
attracting at least some cells with the cell attractant into the inlet and at least partially into the channel;
detecting the presence of the cells in the channel;
wherein allowing the cell attractant to flow through the channel comprises restricting a flow of attractant through the channel by providing a plurality of restrictions in the channel.

16. The method as recited in claim 15, wherein allowing the cell attractant to flow through the channel further comprises, after restricting the flow of attractant through at least one of the plurality of restrictions, allowing the flow to expand into an expansion cavity in the channel.

17. The method as recited in claim 15, wherein allowing the cell attractant to flow through the channel further comprises, after restricting the flow of attractant through each of the plurality of restrictions, allowing the flow to expand into one of a plurality of expansion cavities in the channel.

18. The method as recited in claim 15, wherein the method further comprises, regulating the flow of attractant from the attractant cavity.

19. The method as recited in claim 18, wherein regulating the flow of attractant from the attractant cavity comprises providing a porous medium containing the attractant into the attractant cavity.

20. The method as recited in claim 19, wherein the porous medium containing the cell attractant comprises at least one of a porous silicon, a porous hydrogel, and a porous protein.

21. The method as recited in claim 15, wherein detecting the presence of the cells in the channel comprises providing a plurality of electrodes positioned to contact at least some cells attracted into the channel, wherein contact with the cells provides a detectable variation in an electrical property across at least two of the plurality of electrodes

22. The method as recited in claim 19, wherein the porous medium comprises a hydrogel comprising one or more polymers, and wherein regulating the flow of attractant from the attractant cavity comprises varying the concentration of the one or more polymers of the hydrogel.

23. The method as recited in claim 19, wherein the porous medium comprises a hydrogel comprising one or more cross-linking polymers, and wherein regulating the flow of attractant from the attractant cavity comprises varying the concentration of the one or more cross-linking polymers of the hydrogel

24. The method as recited in claim 23, wherein the one or more cross-linking polymers comprise at least one of PEGDA and a blend of PEGDA and PEGMA.

25. The method as recited in claim 24, wherein regulating the flow of attractant from the attractant cavity comprises regulating a concentration of one of the at least one of PEGDA and the blend of PEGDA and PEGMA.

26. The method as recited in claim 25, wherein regulating the concentration of one of the at least one of PEGDA and PEGMA comprises regulating a concentration of PEGDA to at least 20% and a concentration of PEGMA to at least 1.5%.

27. The method as recited in claim 21, wherein the method further comprises transmitting the detectable variation in the electrical property to a remote receiver.

28. The method as recited in claim 21, wherein the electrical property comprises impedance.

29. The method as recited in claim 15, wherein the cell attractant comprises at least one of EGF and CSF.

30. An implantable attractant dispersing device comprising:
a housing having an outlet for attractant and an attractant cavity; and
a porous medium containing attractant positioned in the attractant cavity and adapted to release attractant out of the outlet.

31. The device as recited in claim 30, wherein the porous medium comprises at least one of a porous silicon and a porous hydrogel.

32. The device as recited in claim 30, wherein the porous medium comprises a hydrogel comprising at least one of PEGDA and a blend of PEGDA and PEGMA.

33. The device as recited in claim 30, wherein the device further comprises a channel positioned between the outlet and the attractant cavity.

34. The device as recited in claim 33, wherein the channel further comprises a plurality of restrictions between the attractant cavity and the outlet.

35. The device as recited in claim 33, wherein the channel further comprises a plurality chambers between the attractant cavity and the outlet.

36. The device as recited in claim 32, wherein the porous medium comprises a hydrogel comprising about 10% to about 30% PEGDA and about 0.5% to about 15% PEGMA.

37. A porous medium for releasing a compound at a desired release rate comprising a hydrogel comprising at least one of PEGDA and a blend of PEGDA and PEGMA.

38. The porous medium as recited in claim 37, wherein the porous medium comprises a hydrogel comprising about 10% to about 30% PEGDA and about 0.5% to about 15% PEGMA.

39. The porous medium recited in claim 37, wherein the compound comprises a chemoattractant.

40. The porous medium recited in claim 39 wherein the chemoattractant comprises a cell attractant.

41. The porous medium recited in claim 40 wherein the chemoattractant comprises at least one of EGF and CSF.
